(19) United States
(12) Patent Application Publication
(10) Pub. No.: US 2025/0121538 A1
(43) Pub. Date: Apr. 17, 2025

(54) Method for connecting two silicone tube sections as well as pharmaceutical media transfer method with such a connection method and apparatus for performing the method

(71) Applicant: RAUMEDIC AG, Münchberg (DE)

(72) Inventors: Philipp GOLLER, Grafengehaig (DE); Michael DRECHSEL, Rehau (DE); Steffen HAGER, Schwarzenbach an der Saale (DE)

(21) Appl. No.: 18/832,813

(22) PCT Filed: Jan. 10, 2023
(86) PCT No.: PCT/EP2023/050449

§ 371 Date: Jul. 24, 2024

(30) Foreign Application Priority Data
Jan. 24, 2022 (DE) 10 2022 200 748.4

(51) Int. Cl. B29C 45/14; A61M 39/12; A61M 39/18; B29C 45/00; B29C 45/17; B29C 65/00; B29C 65/14; B29C 65/48; B29C 65/70; B29K 83/00; B29K 683/00; B29L 23/00

(52) U.S. Cl. CPC B29C 45/14614; A61M 39/12; A61M 39/18; B29C 45/0001; B29C 45/0053; B29C 45/14065; B29C 45/14467; B29C 45/1701; B29C 65/1406; B29C 65/4845; B29C 65/70; B29C 66/1142; B29C 66/5221; B29C 66/71; B29C 2045/0075; B29C 2045/14139; B29K 2083/00; B29K 2683/00; B29L 2023/005; B29L 2023/007

(57) ABSTRACT

In a method for connecting two silicone tube sections (1, 2), end face portions (1a, 2a) of the silicone tube sections (1, 2) to be connected that face each other are positioned in an overmolding mold (13) in such a way that the end face portions (1a, 2a) facing each other abut each other. The end face regions (1a, 2a) abutting each other are overmolded in the overmolding mold (13) by filling an overmolding cavity (14) of the overmolding mold (13) with flowable silicone (15). The flowable silicone (15) is cured. This connection method can be used in a pharmaceutical media transfer method for supplying a pharmaceutical medium (5) from a source reservoir (3) to a target reservoir (4). This media transfer method involves displacing (9) the pharmaceutical medium (5) in a silicone supply tube section of a silicone supply tube (6) as well as cutting (10) the silicone supply tube section for creating a sterile end face portion (1a) as well as cutting a silicone discharge tube section for creating a sterile end face portion (2a). After performing the connection method, the pharmaceutical medium (5) is transferred from the source reservoir (3) to the target reservoir (4) via the connected tube sections (1, 2). For carrying out the method, a device (22) with an exchangeable blade (11), an overmolding mold (13) and a positioning device (11a) are used. The connection method can be carried out in a reliable, reproducible and automated manner with reasonable effort.

The present patent application claims priority of the German patent application DE 10 2022 200 748.4, the content of which is incorporated herein by reference.

The invention relates to a method for connecting two silicone tube sections. Furthermore, the invention relates to a pharmaceutical media transfer method for supplying a pharmaceutical medium from a source reservoir to a target reservoir by using the connection method, an apparatus for performing such a method, a use of a connected silicone tube with tube sections connected to each other at their end faces for supplying media in a pharmaceutical media transfer process as well as a use of a liquid, UV curing silicone material as overmolding material in one of the named methods.

The connection of tube sections in the context of pharmaceutical and biomedical media transfer methods, respectively, is known in the prior art in the context of tube materials which are designed as thermoplastic elastomers. Such methods are known from EP 2 637 839 B1, EP 1 056 970 B1 and WO 2021/118780 A1 as well as EP 3 060 292 B1 and EP 3 603 735 A1. EP 2 999 513 B1 discloses an apparatus for a method for producing a sterile connection of tubes. US 2009/0 243 284 A1 discloses a fluid transfer assembly and methods used for this purpose. DE 10 2019 202 513 A1 discloses an apparatus for forming or molding plastic elements onto surfaces of a semi-finished product.

It is an object of the present invention to develop a method for connecting two silicone tube sections further in such a way that it can be performed in a reliable, reproducible and automated manner with reasonable effort.

According to the invention, this object is solved by a connection method with the features specified in claim 1.

According to the invention, it has been recognized that overmolding end face portions abutting each other of the silicone tube sections to be connected with flowable silicone, which is subsequently cured, allows for a reliable connection solution. As long as appropriately sterilized silicone tube sections are connected, the result is a biocompatible and reliable fluid connection between the connected silicone tube sections.

The flowable silicone may be Liquid Silicone Rubber (LSR, liquid silicone). It may be a LSR material type in the hardness range between Shore A20 to Shore A70.

The flowability of the silicone is such that the overmolding cavity is filled sufficiently. The viscosity of the flowable silicone may thereby be adjusted to a typical cavity size and cavity shape, respectively, of the overmolding cavity.

A silicone tube section in the meaning of the present application is a tube section made from a material, which predominantly consists of silicone. The end face portions of the silicone tube sections abutting each other need not abut each other via an entire tube circumference. This is however preferred.

Curing the flowable silicone is performed by using a UV light irradiation, particularly in the UV-A wavelength range in the range between 315 nm and 400 nm, especially in the range between 315 nm and 380 nm. In principle, curing the flowable silicone by means of UV light irradiation can also be performed by irradiating with another UV wavelength in the range between 100 nm and 350 nm, particularly in the range between 280 nm and 350 nm (UV-B). Thereby, a light source emitting broadband UV light may be used, in which an actually used UV wavelength range is then filtered out.

Another object of the invention is to improve a pharmaceutical media transfer method for supplying a pharmaceutical medium from a source reservoir to a target reservoir in view of its operational and handling reliability.

According to the invention, this object is solved by a media transfer method with the features specified in claim 2. By using the connection methods according to the invention in the pharmaceutical media transfer method, its advantages are particularly evident. Such a media transfer can take place in an entire production chain of a pharmaceutical production process, for example as part of the production process from a batch approach up to a final filling process. The media transfer method can be used in the context of development steps of pharmaceuticals from the laboratory development through scale up intermediate steps up to a mass production. An example for the pharmaceutical media transfer method is a pharmaceutical filling method, in particular the addition of a buffer solution for the pH value regulation in the target reservoir. A sterile container, a process reactor or even a bag may be used as target reservoir.

The pharmaceutical media transfer method may also include a sterilization step for the end face portions of the tube sections created during cutting. Such a sterilization of the end face portions may again be performed by means of a UV light irradiation. Hereby, a UV-C wavelength in the wavelength range between 100 nm and 280 nm, particularly in the range between 100 nm and 200 nm or between 200 nm and 280 nm, can be used.

A cutting according to claim 3 has turned out to be particularly suitable. The cutting may be performed purely mechanically as cold cut, that is without temperature effect. The cutting temperature may be smaller than 70° C., may be smaller than 60° C., may be smaller than 50° C., may be smaller than 40° C. and can even be smaller than 30° C. The cutting temperature is regularly greater than 10° C.

The advantages of an apparatus according to claim 4 correspond to those which were already explained above with reference to the methods. A device, which is already known for the positioning in the context of the connection of TPE tube components, can be used as positioning device.

A mobile configuration according to claim 5 is particularly suitable for the laboratory and clinical use, respectively. Even in a clean room or in a pre-series environment, this mobile configuration can advantageously be used. The connection apparatus can be designed to be mobile. The apparatus performing the connection method can in particular be designed to be mobile. For this purpose, the apparatus can have several castors, which are mounted on the frame of the apparatus. In the mobile configuration, the apparatus can also have a traction drive, for example via at least one electric motor. The connection apparatus can have a self-sufficient energy supply, can also be configured such that it does not depend on external mains connections. The connection apparatus can be designed for battery operation. The connection apparatus can have a set of cutting units, such as blades. The connection apparatus can have a set of exchange overmolding molds. The connection apparatus can have a reading unit for presetting a blade within the blade set and/or for presetting and overmolding mold within the overmolding mold set. This preset can be matched to the tube sections to be connected respectively.

For the corresponding preset, the tubes can have presetting data for the tube sections to be connected, which are provided in coded form for example in form of a QR code. The reading unit can then be designed as QR code reader.

A use of a connected silicone tube according to claim 6 results in advantages, which are already discussed above. It can in particular result in a robust and tear-resistant connection, which simultaneously allows for a reliable media and fluid passage, respectively.

The use of a UV curing silicone material according to claim 7 has turned out to be particularly suitable for the connection method.

The flowable silicone material may be a one-component or two-component material.

A hardness range of the silicone material according to claim 8 has turned out to be particularly suitable for the production of a reliable and simultaneously stable connection.

Different UV wavelengths for curing, on the one hand, and for sterilization, on the other hand, according to claim 9 have turned out to be particularly suitable for the process reliability as well as for the process speed. The UV curing wavelength may be a UV-A wavelength. The UV sterilization wavelength may be a UV-C wavelength.

Embodiments of the invention are described in more detail in the following with reference to the drawing.

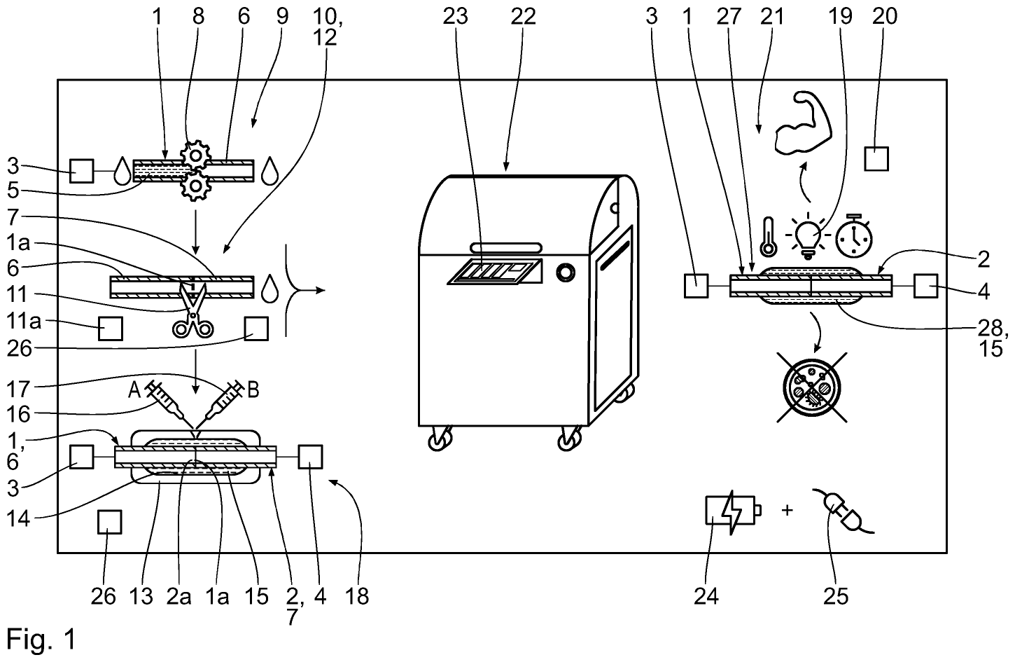

The only FIG. 1 shows a process scheme of a method for connecting two silicone tube sections as well as an apparatus for performing the method.

A method for connecting two silicone tube sections 1, 2 is used in a pharmaceutical media transfer method, in particular in a pharmaceutical discharge method for supplying a pharmaceutical medium from a source reservoir 3 to a target reservoir 4. In principle, the connection method can also be used in another pharmaceutical, biological, medical-technical or even other natural scientific or industrial method.

The silicone tube sections 1, 2 are tube sections made from a material, which predominantly, that is more than 50% by weight, consists of silicone. The silicone material of the silicone tube sections 1, 2 can also consist of more than 60% by weight, more than 70% by weight, more than 75% by weight, more than 80% by weight, more than 85% by weight more than 90% by weight, more than 95% by weight, or even more than 98% by weight silicone.

The source reservoir 3 is thereby in media communication with the silicone tube section 1 and the target reservoir 4 is in media communication with the silicone tube section 2, for example in fluid communication, in particular for guiding a liquid medium.

The pharmaceutical method goes as follows:

First, a pharmaceutical medium 5 is provided in the source reservoir 3 including a silicone supply tube 6 being in media communication with the source reservoir 3. The silicone tube section 1 is part of the silicone supply tube 6. Additionally, the target reservoir 4 including a silicone discharge tube 7 being in media communication with this reservoir is provided. The silicone tube section 2 is part of the silicone discharge tube 7.

Now, the pharmaceutical medium 5 is displaced in a silicone supply tube section of the silicone supply tube 6 in particular towards the source reservoir 3, which is depicted at the top left of FIG. 1, where the pharmaceutical medium 5 is displaced from a right half of the shown section of the silicone supply tube 6 by using a displacement mechanism 8. FIG. 1 thus shows a displacement step 9 of the filling method at the top left.

In a cutting step 10 (cf. FIG. 1, middle left) of the filling method, the silicone supply tube section of the silicone supply tube 6 is then cut with a cutting unit 11 for creating a sterile end face portion 1a of the silicone supply tube section. In the same way, a silicone discharge tube section of the silicone discharge tube 7 is cut with the cutting unit 11 for creating a sterile end face portion 2a of the silicone discharge tube section in a further cutting step 12, which is also depicted in FIG. 1 middle left. The cutting unit 11 may be an exchangeable blade.

The two cutting steps 10, 12 for cutting the tubes 6, 7 can be performed at the same time with the same cutting unit 11. For this purpose, the two tubes 6, 7 can be arranged in parallel to each other.

The two tube sections, as the silicone supply tube section, on the one hand, and the silicone discharge tube section, on the other hand, represent the two silicone tube sections 1, 2 to be connected after the cutting steps 10, 12.

Cutting the silicone supply tube section 6 and the silicone discharge tube section 7 is performed with the cutting unit 11 at a cutting temperature, which is smaller than 80° C. This temperature can be smaller than 70° C., can be smaller than 60° C., can be smaller than 50° C., can be smaller than 40° C., can be smaller than 30° C. The cutting steps 10, 12 are each a cold cut, which is performed purely mechanically, that is not under the influence of heat on the silicone material of the silicone tube sections.

A sterilization of the two end face portions 1a, 2a can be performed in the context of the filling method by using a UV sterilization irradiation of these end face portions 1a, 2a.

After the cutting steps 10, 12, the two silicone tube sections 1, 2 are positioned in an overmolding mold 13 with the end face portions 1a, 2a facing each other in such a relation to each other that the end face portions 1a, 2a abut each other. For positioning the silicone tube sections 1, 2 to be connected, a positioning device that is in principle already known from the prior art can be used. Such a positioning device is schematically indicated in FIG. 1, middle left, at 11a.

Now, the end face portions 1a, 2a abutting each other are overmolded in the overmolding mold 13 by filling the overmolding cavity 14 of the overmolding mold 13 with flowable silicone 15.

The flowable silicone may be a two-component (2C) material with components A and B, which are supplied to the overmolding cavity 14 with dosing units 16, 17 via separate feed dosing channels. Alternatively, the flowable silicone can also be a one-component (1C) material. As long as the flowable silicone 15 is a one-component material, this component contains a base polymer, a catalyst and a cross-linking agent. As long as the flowable silicone 15 is a two-component material, one of the two components can contain a base polymer and a cross-linking agent and the other component can contain a catalyst.

The dosing units 16, 17 represent a feeding device for the flowable silicone 15 as overmolding material.

In the pharmaceutical filling method, in particular in the connection method, a flowable, UV curing silicone material in form of the flowable silicone 15 as overmolding material is used.

FIG. 1 shows, at the bottom left, an overmolding step 18 of the connection method after the preceding positioning step which is not depicted in detail.

After overmolding 18, curing the flowable silicone is performed by irradiating the flowable silicone 15 via a UV lamp 19 with a UV curing wavelength. The UV curing wavelength may differ from the UV sterilization wavelength. The cured silicone material resulting from the flowable silicone 15 may have a hardness in the range Shore A 40 to Shore A 60.

A curing irradiation and a sterilization irradiation may be performed in the course of the pharmaceutical filling method in the same process step.

An irradiation period during curing and cross-linking of the flowable silicone 15, respectively, is in the range between 10 s and 120 s, for example between 60 s and 120 s. A cross-linking wavelength is in the UV-A range, that is in a wavelength range between 315 nm and 400 nm.

During sterilization, an irradiation period is in the range between 5 s and 1 min, for example between 10 s and 50 s or between 15 s and 45 s. A sterilization irradiation wavelength is in the UV-C range, that is in a wavelength range between 200 nm and 280 nm, for example in a range between 240 nm and 260 nm.

For the cross-linking irradiation, on the one hand, and the sterilization irradiation, on the other hand, two different UV sources can be used. Alternatively, it is possible to work with a collective UV source, for which the respective irradiation wavelengths for cross-linking/curing as well as for sterilization are set by corresponding filters.

The cross-linking irradiation period may be longer than the sterilization irradiation period. Alternatively, the sterilization irradiation period may also be longer than the cross-linking irradiation period. Both irradiation periods may also be equally long.

The cross-linking/curing irradiation is performed without overlapping in time with the sterilization irradiation period. Regularly, the sterilization irradiation is performed in the course of the connection method before the cross-linking/curing irradiation.

The sterilization and curing irradiation, respectively, are performed by a control unit 20 in a temperature and/or time controlled manner. A corresponding curing step 21 is depicted at the top right of FIG. 1.

The middle of FIG. 1 shows a connection apparatus 22 for performing the pharmaceutical filling method and in particular for performing the connection method.

The connection apparatus 22 includes the exchangeable blade 11 as well as the overmolding mold 13. The connection apparatus 22 may have a magazine with several exchangeable blades 11 or exchange blades, respectively, which may be used selectively and particularly in an automated way. With one blade, 10 to 100 cutting processes can for example be performed. The connection apparatus 22 may have a set of blades 11. The respective overmolding mold 13 may also be tailored to the tube sections 1, 2 to be connected.

The connection apparatus 22 may have a set of overmolding molds 13 particularly for receiving different outer diameters of silicone tube sections of the type of the silicone tube sections 1, 2.

The overmolding mold 13 may be designed as exchange overmolding mold. The connection apparatus 22 may have a mold magazine, in which a plurality of such overmolding molds have in particular several, selectable sizes of overmolding cavities. Exchanging the exchange overmolding molds may be performed in an automated manner. For example, 500 to 100,000, in particular 10,000 to 25,000 overmolding processes can be performed per overmolding mold. The connection apparatus 22 may store between 3 and 12 overmolding mold sizes, which are in particular tailored to pharmaceutical standard sizes of the outer diameters of the silicone tube sections 1, 2.

The overmolding mold 13 may be received in a mold receptacle of the connection apparatus 22, which has a contour, which is complementary to the contour of the received overmolding mold. This ensures that the overmolding mold is prevented from twisting in the mold receptacle on the one hand and that the overmolding mold is on the other hand properly oriented and positioned in the mold receptacle, respectively.

Between the overmolding mold and the mold receptacle, there may be a signal connection, which ensures that the correct overmolding mold is used for a current connection task. This can for example be achieved by a plurality of contact pins of the mold receptacle, between which respective conductive connections are established via the overmolding mold when selected correctly.

The overmolding mold 13 may be made from a polymeric material, such as PMMA.

An operation period and a number of utilization cycles of the respective blades 11 may be preset and documented in the control unit 20, which can also be used to control the blades 11.

The control unit 20 may additionally be used for the documentation of the operation of the respective UV lamp for curing on the one hand and for sterilizing on the other hand. Each connection process can thereby be evaluated in the control unit 20.

In particular a radiation intensity, an irradiation period as well as a temperature in particular of the flowable silicone 15 during the curing process and the end face portions 1a, 2a during the sterilization process, respectively, can thereby be preset and documented.

After connecting has been done, filling the pharmaceutical medium from the source reservoir 3 through the silicone tube sections 1, 2 being now connected to each other to the target reservoir 4 takes place. The pharmaceutical medium is for example a buffer solution for pH value regulation.

The connection apparatus 22 may have mobile configuration. The connection apparatus 22 has a display/operating unit 23, which may be designed as touchscreen and which is in signal connection with the control unit 20.

The connection apparatus 22 may be designed to be mobile. The connection apparatus 22 may be designed for battery operation 24 and/or for mains operation 25.

The connection apparatus 22 may have a reading unit 26 for presetting the respective blades 11 and/or for presetting the respective overmolding mold 13, which is schematically shown in the middle left and bottom left of FIG. 1 respectively. The reading unit 26 may thereby read a code on the respective blade 11 and/or on the respective overmolding mold 13, such as a QR code. Alternatively or additionally, the reading unit 26 may read a corresponding code on at least one of the silicone tube sections 1, 2, which may in turn be designed as QR code.

A silicone tube 27, being readily connected, with the two connected, sterile end face portions 1a, 2a of the silicone tube sections 1, 2, which are connected to each other by overmolding 28, is used for media supply in a pharmaceutical filling process.

1-9. (canceled)

10. Method for connecting two silicone tube sections, comprising:
sterilizing end face portions of the silicone tube sections to be connected by means of UV light irradiation with a UV sterilization wavelength;
positioning the end face portions of the silicone tube sections to be connected that face each other in an overmolding mold such that the end face portions facing each other abut each other;
overmolding the end face portions abutting each other in the overmolding mold by filling an overmolding cavity of the overmolding mold with flowable silicone; and
curing the flowable silicone by UV light irradiation by means of a UV light irradiation with a UV curing wavelength;
wherein the UV curing wavelength differs from the UV sterilization wavelength.

11. Pharmaceutical media transfer method for supplying a pharmaceutical medium from a source reservoir to a target reservoir, comprising:
providing the pharmaceutical medium in the source reservoir including a silicone supply tube being in media communication with the source reservoir;
providing the target reservoir including a silicone discharge tube being in media communication with the target reservoir;
displacing the pharmaceutical medium in a silicone supply tube section of the silicone supply tube;
cutting the silicone supply tube section of the silicone supply tube for creating a sterile end face portion of the silicone supply tube section;
cutting a silicone discharge tube section of the silicone discharge tube for creating a sterile end face portion of the silicone discharge tube section;
connecting the silicone supply tube section with the silicone discharge tube section with a method according to claim 1, wherein the silicone supply tube section on the one hand and the silicone discharge tube section on the other hand represent the two silicone tube sections to be connected by the method; and
transferring the pharmaceutical medium from the source reservoir to the target reservoir via the connected tube sections.

12. Pharmaceutical media transfer method according to claim 11, characterized in that the cutting of the silicone supply tube section and/or the silicone discharge tube section is performed at a cutting temperature that is less than 80° C.

13. Apparatus for connecting two silicone tube sections and for performing a method according to claim 10, comprising:
a UV source for sterilizing the end face portions of the silicone tube sections to be connected by means of a UV light irradiation with a UV sterilization wavelength;
a cutting unit for cutting at least one of the silicone tube sections,
an overmolding mold including a feeding device for the flowable silicone as overmolding material,
a positioning device for positioning the silicone tube sections to be connected; and
a UV source for curing of the flowable silicone by UV light irradiation by means of a UV light irradiation with a UV curing wavelength.

14. Apparatus according to claim 13, wherein the apparatus has a first UV source for sterilizing the end face portions of the silicone tube sections to be connected by means of a UV light irradiation with a UV sterilization wavelength and a second UV source for curing the flowable silicone by means of a UV light irradiation with a UV curing wave-length.

15. Apparatus according to claim 13, wherein the apparatus has a collective UV source configured such that the respective irradiation wavelengths for sterilizing the end face portions of the silicone tube sections to be connected and for curing the flowable silicone are settable by corresponding filters.

16. Apparatus according to claim 13, characterized by a mobile configuration for the laboratory use, the clinical use, the use in a clean room environment or for the use in a pre-series environment;
wherein the apparatus is designed to be mobile and has several castors, which are mounted on a frame of the apparatus; and
wherein the apparatus in particular has a traction drive, in particular an electric motor.

17. Apparatus according to claim 13, further comprising:
a set of blades; and
a set of overmolding molds;
wherein the tubes have presetting data for the tube sections to be connected, namely the silicone supply tube and the silicone discharge tube; and
wherein the apparatus further has a reading unit for presetting a blade within the set of blades and/or for presetting an overmolding mold within the set of overmolding molds, matched to the tube sections to be connected respectively.

18. Apparatus according to claim 17,
wherein the blades of the set of blades and the overmolding molds of the set of overmolding molds have a code, in particular a QR code, respectively;
wherein the presetting data of the tubes for the tube sections to be connected are provided in form of codes, in particular QR codes; and
wherein the reading unit is designed as a code reader, in particular as a QR code reader.

19. A method of using a liquid, UV curing silicone material as overmolding material in a method according to claim 10, which is designed in such a way that a UV curing wavelength used in curing the silicone material differs from a UV sterilizing wavelength used in the context of a sterilizing step of the silicone material.

20. Method according to claim 19, wherein the UV curing of the silicone material in cured state has a hardness in the range of Shore A40 to Shore A60.
